(19) United States
(12) Patent Application Publication
(10) Pub. No.: US 2026/0273293 A1
(43) Pub. Date: Sep. 17, 2026

(54) Automated External Defibrillator Device with Synchronized Cardioversion and Methods of Use

(71) Applicant: HeartHero, Inc., Denver, CO (US)

(72) Inventors: Gary Montague, Denver, CO (US); Marina V. Brockway, Shoreview, MN (US); Brian P. Brockway, Shoreview, MN (US)

(21) Appl. No.: 19/474,020

(22) PCT Filed: Apr. 10, 2024
(86) PCT No.: PCT/US2024/023895

§ 371 Date: Oct. 9, 2025

Related U.S. Application Data
(60) Provisional application No. 63/495,309, filed on Apr. 11, 2023.

(51) Int. Cl. A61N 1/39

(52) U.S. Cl. CPC A61N 1/3925; A61N 1/3904; A61N 1/3987; A61N 1/3993

(57) ABSTRACT

An automated external defibrillator system includes shock generating electronics, an electrocardiogram (ECG) monitoring circuitry, and a controller for controlling operations of the shock generating electronics and ECG monitoring circuitry. The controller is further configured for receiving and analyzing ECG signals from the ECG monitoring circuitry, predicting timing of QRS complexes associated with the ECG signals, and directing the shock generating electronics to deliver an electrical shock in synchrony with the timing of the QRS complexes so predicted. In embodiments, the controller directs the shock generating electronics to deliver the electrical shock in synchrony with an R wave of the QRS complexes so predicted. In embodiments, the controller analyzes for ventricular fibrillation (VF) associated with the ECG signals, predicts a trend in VF peaks, and directs the shock generating electronics to deliver the electrical shock in synchrony with a VF peak according to the predicted trend.

### CROSS-REFERENCE TO RELATED APPLICATIONS

This application is the national phase of, and claims priority to, International Application No. PCT/US 2024/023895, filed Apr. 10, 2024, titled “Automated External Defibrillator Device with Synchronized Cardioversion and Methods of Use,” which claims priority to U.S. Provisional Patent App. No. 63/495,309, filed Apr. 11, 2023, titled “Automated External Defibrillator Device with Synchronized Cardioversion and Methods of Use.” The present application also relates to U.S. patent application Ser. No. 15/847,826, filed Dec. 19, 2017, titled “Automatic External Defibrillator Device and Methods of Use,” now issued as U.S. Pat. No. 11,103,718, which claims priority to U.S. Provisional Patent App. No. 62/436,208, filed Dec. 19, 2016, titled “Automatic External Defibrillator Device and Methods of Use.” All of the above referenced applications are incorporated hereby in their entirety by reference.

### FIELD OF THE INVENTION

The present invention relates to automated external defibrillators (AEDs). In particular, but not by way of limitation, the present invention relates to AEDs with features to enable synchronized cardioversion.

### DESCRIPTION OF RELATED ART

86 million Americans have risk factors for sudden cardiac arrest (SCA), while 12 million are at high risk. Cardiac events represent more deaths in America than breast, lung, colon and prostate cancer combined. More than 360,000 sudden cardiac arrest (SCA) occur outside of the hospital each year. According to the American Heart Association, nearly 70 percent of these SCAs occur at home, out of reach of the lifesaving shock of an AED.

As each minute passes following a sudden cardiac arrest, the chances of survival decrease significantly. If an AED is not applied within 10 minutes of a SCA event, chances of survival decrease to less than 1%.

One approach to increasing the chance of survival for SCA sufferers is to make AEDs more readily available and accessible for more people. However, the AEDs currently available on the market tend to be heavy, not portable, expensive, and intimidating to use for people without medical training. For example, the above-referenced patent, provides a possible solution to overcome the availability and accessibility problem by providing a compact AED device suitable for portability.

Thus, there is a need for an improved, portable AED with additional functionalities.

### SUMMARY OF THE INVENTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

In an embodiment, an automated external defibrillator system integrated with a shock synchronization function is described.

In a further embodiment, an associated method of operating an automated external defibrillator system is also described herein.

In embodiments, an automated external defibrillator system includes shock generating electronics, an electrocardiogram (ECG) monitoring circuitry, and a controller for controlling operations of the shock generating electronics and the ECG monitoring circuitry, the controller including non-transitory memory for storing software instructions and a processor for executing software instructions stored on the non-transitory memory. The controller may be configured for receiving and analyzing ECG signals from the ECG monitoring circuitry, predicting timing of QRS complexes associated with the ECG signals, and directing the shock generating electronics to deliver an electrical shock in synchrony with the timing of the QRS complexes so predicted.

In embodiments, the controller is configured for directing the shock generating electronics to deliver the electrical shock in synchrony with an R wave of the QRS complexes so predicted.

In embodiments, the controller is further configured for analyzing for ventricular fibrillation (VF) associated with the ECG signals, predicting a trend in VF peaks associated with the ECG signals, and directing the shock generating electronics to deliver the electrical shock in synchrony with a VF peak according to the trend in VF peaks so predicted.

In embodiments, a method for operating an automated external defibrillator system is disclosed. The method includes receiving and analyzing electrocardiogram signals from a patient through an electrocardiogram monitoring function in the automated external defibrillator system, predicting timing of QRS complexes associated with the ECG signals, and delivering an electrical shock to the patient in synchrony with the timing of the QRS complexes so predicted.

In embodiments, a method for operating an automated external defibrillator system includes receiving electrocardiogram (ECG)signals from a patient through an ECG monitoring function in the automated external defibrillator system, analyzing for ventricular fibrillation (VF) associated with the ECG signals, predicting a trend in VF peaks associated with the ECG signals, and delivering the electrical shock to the patient in synchrony with a VF peak according to the trend in VF peaks so predicted.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF DRAWINGS

FIG. 1 shows an example graphic of QRS complex data, with the various markers indicated.

FIG. 2 shows a block diagram of an exemplary AED with integrated VT and/or VF synchronization functions, in accordance with an embodiment.

FIG. 3 shows an exemplary process flow for the operation of an AED with integrated VT synchronization, in accordance with an embodiment.

FIG. 4A shows an example ECG readout for a patient with normal sinus rhythm

FIG. 4B shows an example ECG readout for a patient with ventricular fibrillation (VF).

FIG. 5 shows an exemplary process flow for the operation of an AED with integrated VF synchronization, in accordance with an embodiment.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION OF THE INVENTION

Existing AEDs (such as described in the patent references above) provide monitoring a patient's electrical signal from the heart, then determining the presence of a shockable rhythm. In certain cases, the detection of ventricular fibrillation (VF) or pulseless ventricular tachycardia (VT) may be an indication of a shockable rhythm.

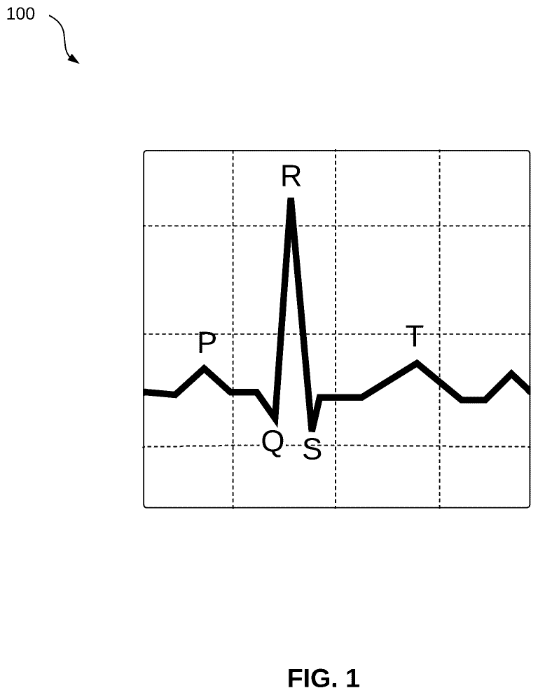

Beyond the determination of the presence of a shockable rhythm, some AEDs are connected with an ECG to monitor the QRS complex of the heart rhythm. An example of an electrical signal corresponding to a heartbeat, with the relevant points of note indicated, is shown in FIG. 1 (Image from

https://www.healio.com/cardiology/learn-the-heart/ecg-review/ecg-interpretation-tutorial/qrs-complex, retrieved 2023-01-23).

FIG. 1 shows a simplified representation 100 of a QRS complex corresponding to a heartbeat. As shown in FIG. 1, a QRS complex generally includes a P wave (small peak indicated with a letter P), a Q wave (valley indicated with a letter Q), an R wave (peak indicated with the letter R), an S wave (valley indicated with a letter S), and a T wave (small peak indicated with the letter T). In most cases, the P wave represents depolarization of the left and right atria, corresponding to atrial contraction. The Q, R, and S waves are associated with ventricular depolarization, and the QRS complex generally occurs just before ventricular contraction.

It is noted that each square in the graphic in FIG. 1 is drawn to be larger than a standard electrocardiogram (ECG) output. Whereas each square in a standard ECG output corresponds to 40 milliseconds along the horizontal axis, each square in FIG. 1 corresponds to approximately 100 milliseconds for illustrative clarity only.

Generally, defibrillation refers to high energy, unsynchronized shock, whereas cardioversion refers to low energy, synchronized shock. Defibrillation is indicated for Pulseless ventricular tachycardia, ventricular fibrillation, and cardiac arrest resulting either from or causes ventricular fibrillation. Cardioversion (a.k.a. synchronized cardioversion) may be used, for example, to convert the heart rhythm from arrhythmia to normal, particularly for Unstable atrial fibrillation (AF), atrial flutter (types I and II), atrial tachycardia, ventricular tachycardia (VT) with a pulse, and supraventricular tachycardia (SVT). Still further improvement in the odds of a favorable outcome with AED application may be obtained by the use of synchronized electrical cardioversion to synchronize the application of electrical shock with the monitored electrical signals corresponding to the heart rhythm (see, for example, Goyal, et al., “Synchronized Electrical Cardioversion,”National Center for Biotechnology Information, NIH National Library of Medicine, 2022-05-22, https://www.ncbi.nlm.nih. gov/books/NBK482173/#:~:text=Synchronized%20cardioversion%20is%20a% 20procedure,threatening%20or%20unstable%20tachycardic%20arrhythmia., retrieved 2023-02-16).

For instance, research has shown that application of an electrical shock with the T wave may result in ventricular fibrillation (VF), thus inducing cardiac arrest in a patient who originally had a pulse prior to the application of the electrical shock. With the QRS complex data, a user may manually or automatically (using software integrated into the ECG, for example) aim to synchronize the delivery of electrical shock with the R wave.

Defibrillators with so-called “sync” features exist, with which the user may initiate a shock delivery process, and the defibrillator awaits the following R wave after the initiation to deliver the actual shock. However, such defibrillators with sync features generally require the analysis of ECG data over a span of several seconds (e.g., multiple QRS cycles from the initiation of shock delivery process, with each QRS cycle lasting 80 to 100 milliseconds). When every second counts towards the patient's survival during a cardiac event, such additional cycles of analysis may result in wasted time before the application of the potentially life-saving electrical shock. Further, even with sync-enabled defibrillators, the sync function generally acts as an add-on to the AED functionality, implemented as a separate timing circuitry outside of the normal shock delivery processes.

### AED With VT Sync

In an embodiment, an AED includes monitoring and analysis circuitry as a part of the electrical shock delivery functionality such that no separate input from a user (e.g., pressing of a “sync” button) is necessary to enable synchronized cardioversion. The QRS complex monitoring and analysis is initiated upon application of the AED cardiac pads on the patient, as a normal course of AED operations. That is, shock delivery is timed for the peak of the R wave in a given QRS cycle, as soon as shockable rhythm is detected and AED determines the delivery of an electrical shock is warranted, such that the VT sync function is integrated into the shock delivery operations of the AED.

In certain embodiments, such an AED with integrated VT sync functions includes low latency, high sensitivity monitoring of patient heart rhythms, using the same circuitry and analysis processes used in the determination of a shockable rhythm. In some cases, the heart rhythm analysis occurs at rates fast enough to monitor heart rhythms to take multiple measurements within a given QRS cycle in order to anticipate the highest point of the R wave within the given QRS cycle being analyzed.

Additionally, in embodiments, an AED with VT sync functions exhibits low latency from QRS detection to the communication of the detection to the shock delivery mechanism. As an example, a maximum delay from detection of a true R wave peak and the communication of the true R wave peak detection to the shock delivery mechanism is less than 35 milliseconds (e.g., less than a standard ECG readout time interval, or less than half of an average QRS cycle). In an example, the latency of may be less than 20 milliseconds. In certain implementations, the median latency may be 16 milliseconds.

Further, in embodiments, the sensors, firmware, and/or software provide sufficient sensitivity to track trends in the QRS complex and anticipate the peak of the R wave to sync with the shock delivery. For instance, the AED with sync functions includes sensors with sufficient sensitivity to detect electrical signals associated with heart rhythms and provide data of sufficient precision to enable accurate detection of the peak of the R wave, communication of the R wave detection to the shock delivery mechanism, and synchronization of the shock delivery with the immediately following R wave. In certain embodiments, the AED anticipates the timing of the next R wave based on the observation of multiple cycles of heartbeats (and QRS complexes). For example, having observed multiple cycles of heartbeats during the initial patient monitoring in determining the presence of shockable rhythm, the processing functions (e.g., in a microprocessor onboard the AED) may anticipate the average length of time between the P wave and the subsequent Q wave to time the delivery of the electrical shock. Alternatively or additionally, the analysis may include various techniques known in the data analysis arts, such as pattern matching and/or event detection using data libraries and/or machine learning training sets integrated into the processing functions of the AED. In a further embodiment, the sensing and processing capabilities of the AED with sync functions are able to identify the T wave in the QRS complex and enable delivery of the electrical shock to avoid the T wave and/or cardiac repolarization. In certain embodiments, no user intervention, such as the pressing of a sync button, is required to perform the synchronized cardioversion with the sync-enabled AED.

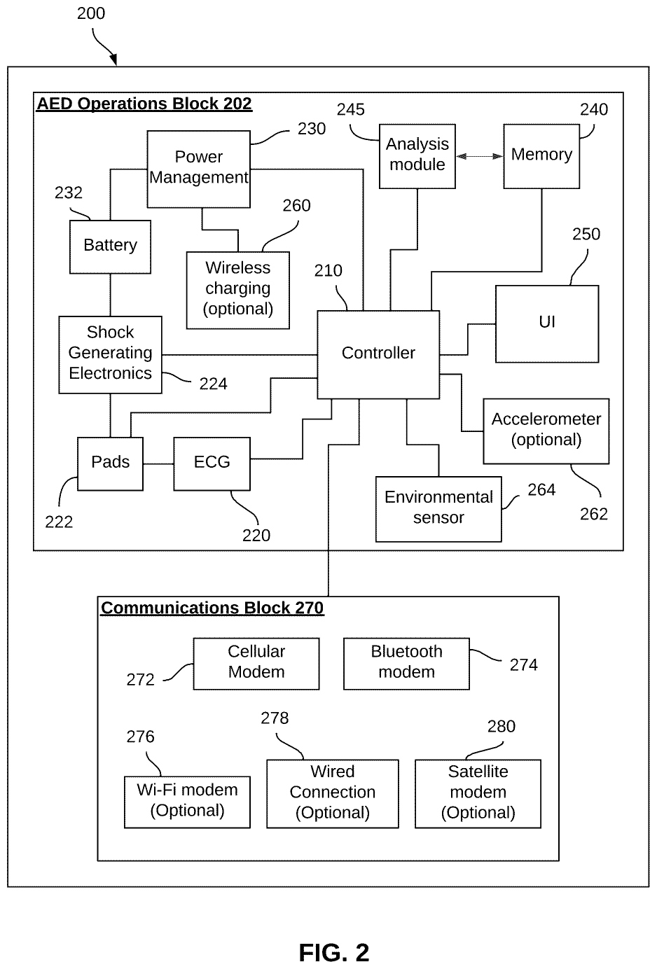

FIG. 2 shows a block diagram of an exemplary AED with integrated VT and/or VF synchronization functions. In particular, FIG. 2 shows an exemplary AED 200 including an AED operations block 202 and a communications block 270, in accordance with an embodiment. AED operations block 202 includes various components that enable AED 200 to generate and deliver, within regulatory guidelines, an electric shock to a person experiencing a cardiac event such as SCA. As shown in the embodiment illustrated in FIG. 2, AED operations block 202 includes a controller 210, which controls a variety of components including an electrocardiogram (ECG) monitoring circuitry 220, which is in turn connected with cardiac pads 222. Cardiac pads 222 are configured for attachment to specific locations on a patient experiencing a cardiac event for delivering an electric shock to the patient. In embodiments, cardiac pads 222 may include additional functionality, such as a medicine delivery mechanism, (e.g., a transdermal patch impregnated with a medicine, a microneedle array imbued with a known dose of a medication, or a blister pack containing a medication).

In embodiments, cardiac pads 222 are configured to receive ECG signals from the patient in addition to administering the electric shock. In some embodiments, cardiac pads 222 are waterproof. Cardiac pads 222 may be configured for reuse a predetermined number of times, after which the cardiac pads are replaced. The ECG signals are transmitted from cardiac pads 222 to ECG monitoring circuitry 220, which is communicatively coupled with controller 210.

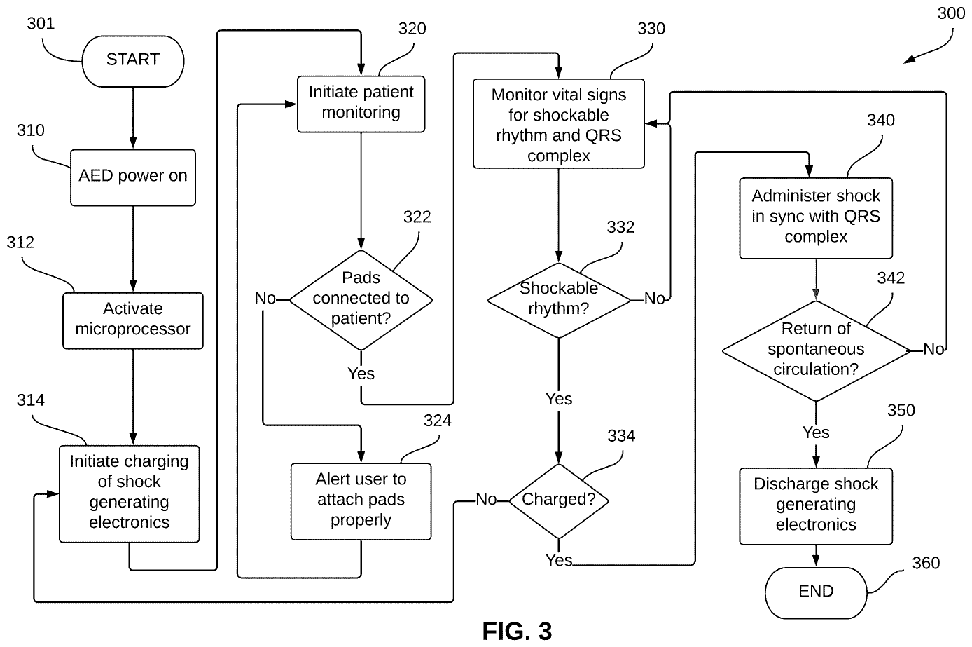

Shock generating electronics 224 are configured to charge a capacitor under control of controller 210. In embodiments, AED 200 includes a flat square capacitor for fitting within the case or enclosure of AED 200, while in other embodiments the charge storing capacitor may be customized to fit certain portions of the AED (e.g., a handle portion of AED 300 shown in FIG. 3). In embodiments, the shock generating electronics 224 are configured to provide a voltage waveform that is between approximately 120 and 200 Joules in total energy. In certain embodiments, the shock generating electronics 224 include a quad-phasic truncated exponential power stage that is configured to produce a shock suitable for defibrillation while reducing the complexity and size of the capacitor.

Controller 210 may include, for example, non-transitory memory for storing software instructions. The non-transitory memory may be communicatively coupled with a processor (e.g., microprocessor) for executing software instructions stored on the non-transitory memory. Software instructions may include, for instance, workflow information for operating AED 200 including VT and/or VF monitoring, as described herein. Controller 210 is also connected with a memory 240, which stores information regarding AED 200, such as use history, battery status, shock administration and CPR protocols, and other information (e.g., stored in look-up tables) used in the operation of AED 200. In embodiments, memory 240 may cooperate with an analysis module for further analyzing the patient heart rhythms for the presence of shockable rhythm, QRS complex prediction, and/or further analysis according to data stored in memory 240. Memory 240, may, in some embodiments, be used by controller 210 to instruct a user on CPR and/or shock administration via communications block 270. In embodiments, controller 210 is a single microcontroller or single microprocessor controlling all aspects of the AED operation.

AED operations block 202 includes a power management block 230, which is also controlled by controller 210 in embodiments. Power management block 230 comprises power management circuitry configured for managing the power consumption by various components within AED operations block 202. For instance, power management block 230 monitors a charge status of a battery 232, which provides electrical power to shock generating electronics 224. Power management block 230 provides instructions for controlling the on/off status of all electrical components of AED 200 via controller 210 so as to minimize power consumption while AED 200 is not being used. For example, power management block 230 is configured to completely power down ECG monitoring circuitry 220 and shock generating electronics 224 when the AED is not being used. In embodiments, power management block 230 is configured to provide a signal to a user via controller 210 for notifying the user of a charge status of battery 232.

A user-interface (UI) 250 is communicatively coupled with controller 210. In embodiments, UI 250 provides a simple user interface that minimizes space and electrical power requirements. For example, UI 250 may not include a screen or a graphic user interface, such as a liquid crystal display (LCD) or touchscreen, if the size and complexity of AED should be reduced. UI 250 may include a speaker to provide audible sounds (e.g., beeps) and/or voice prompts for aiding the AED user.

In certain embodiments, the UI features may be eliminated to further reduce the size and weight of the AED.

In certain embodiments, AED operations block 202 may further include one or more sensors for monitoring the physical state and/or environmental conditions of the AED, such an optional accelerometer 262, an environmental sensor 264 (e.g., for sensing temperature, humidity, etc. surrounding the AED, which conditions may affect the operations of the AED), and others.

Still referring to FIG. 2, AED 200 includes a communications block 270, also controlled by controller 210. Communications block 270 provides connections to external systems and entities outside of the AED, such as emergency medical services, hospital emergency rooms, physicians, electronic health record systems, as well as other medical equipment, such as ventilators and an external ECG. Communications block 270 includes at least one of the following communication features: a cellular modem 272, a Bluetooth® modem 274, a Wi-Fi modem 276 for providing wireless connection to and from an external device. The various communication modes within communications block 270 are configured to comply with regulatory guidance related to wireless technology, such as coexistence, security, and electromagnetic compatibility. By having a single controller (e.g., a microprocessor) control communications block 270 within AED 200, the circuit design and firmware configuration of AED 200 is greatly consolidated over other AEDs with multiple processors, while enabling a reduction in power consumption of the device.

Particularly related to VT synchronization, the monitoring and analysis circuitry may be integrated into AED operations block 202, for example, as part of controller 210 and/or ECG monitoring circuitry 220. In an embodiment, the non-transitory memory within controller 210 may be further configured to store instructions to enable ECG monitoring circuitry 220 to monitor and analyze the QRS complexes of the heart rhythms obtained from the patient. In examples, ECG monitoring circuitry 220 may incorporate analysis software that has been trained by machine learning on a database of QRS complex examples including clinical and/or synthetic training data sets for healthy and cardiac patients.

For instance, ECG monitoring circuitry may be configured to monitor and analyze the incoming heart rhythms of the patient with a latency of less than 20 milliseconds. Further, ECG monitoring circuitry 220 and controller 210 may be configured to cooperate such that the ECG monitoring circuitry and/or processor within controller 210 may receive the heart rhythms, analyze the heart rhythms to predict the upcoming QRS complex timing to enable the delivery of the electrical shock from shock generating electronics 224 to coincide with the next anticipated R wave. In embodiments, the monitoring, analysis, and timing of the shock delivery may be automated such that no further input from the user is needed once the shock delivery sequence has been initiated. The integration of the VT synchronization within the operations of the AED is particularly advantageous as the ECG monitoring and AED operations circuitry are used to time the shock delivery according to the anticipated QRS complex timing, without requiring additional equipment or user input.

An exemplary process flow for the operation of an AED with VT synchronization is shown in FIG. 3. As shown in FIG. 3, a process 300 begins with a start step 301. In a step 310, the AED is powered on, for example, by a user pressing an AED power button or by accessing the cardiac pads. The microprocessor within the AED controller (e.g., controller 210) is activated in a step 312, and the microprocessor initiates the charging protocol for the shock generating electronics in a step 314. The AED then initiates the patient monitoring in a step 320. A decision 322 is made to determine whether the cardiac pads are correctly connected (i.e., attached) to the patient such that the AED is receiving the appropriate heart rhythm data. If decision 322 is NO, then the user is alerted in a step 324 (e.g., by an audible instruction or visible cue via UI 250) to ensure the cardiac pads are properly attached to the patient, and the process returns to step 320. If decision 322 determines the pads are correctly attached to the patient, then process 300 proceeds to a step 330 to monitor the vital signs (including heart rhythms) of the patient for shockable rhythm and QRS complex timing. If shockable rhythm is detected in a decision 332, then process 334 proceeds to a decision 334 to determine if the shock generating electronics of the AED are appropriately charged. If decision 332 determines no shockable rhythm has been detected, process 300 returns to step 330 to continue to monitor the patient vital signs.

If decision 334 determines the electronics have not been appropriately charged, process 300 returns to step 314 to charging the shock generating electronics. If decision 334 determines the electronics are charged and ready to provide shock, then a shock is administered in synchrony with the desired point in the next QRS complex (e.g., with or immediately preceding the R wave) in a step 340. A decision 342 is then made whether spontaneous circulation has resumed. If a determination is made in decision 342 that spontaneous circulation has not yet returned, then process 300 returns to step 330. If spontaneous circulation has returned as a result of the shock administration, then the shock generating electronics are discharged in a step 350, and process 300 is terminated in an end step 360.

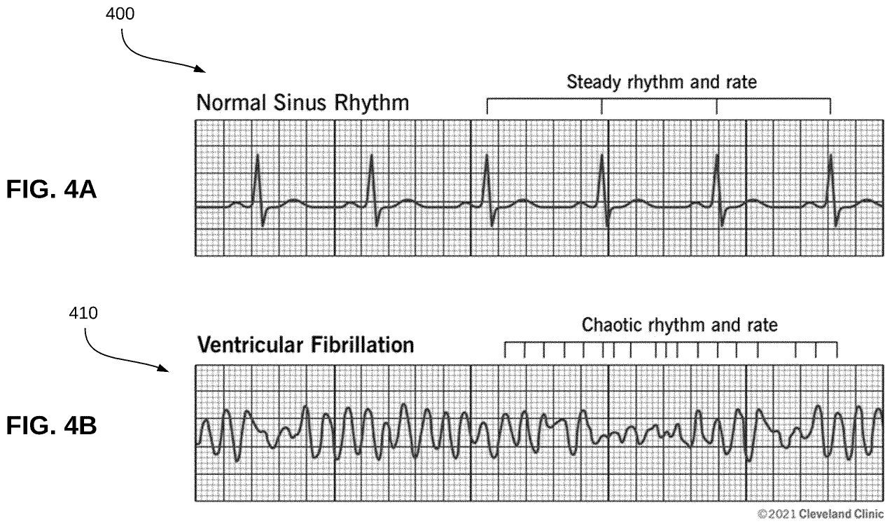

### AED With VF Sync

Whereas synchronized cardioversion is commonly indicated for VT and supraventricular tachycardia (SVT), the conventional rule of thumb is that you cannot synchronize the shock delivery timing for VF due to the chaotic heart rhythm observed during VF. That is, synchronization of the shock delivery with the ECG reading, whether automatically or manually, is considered to be not possible, as the rhythm and rate of the heart rhythm during VF is generally characterized as being chaotic, as illustrated in FIGS. 4A and 4B. In particular, FIGS. 4A and 4B compares the ECG output from a patient with normal sinus rhythm and another patient with VF (Cleveland Clinic, 2021, https://my.clevelandclinic.org/health/diseases/21878-ventricular-fibrillation, retrieved 2023-01-26). It is noted that each small square (generally a 1 millimeter by 1 millimeter square) in the ECG charts above corresponds to a 40 millisecond duration, such that a group of five small squares represents 200 milliseconds, and a group of 25 small squares represents one second in time.

While the National Institute of Health provides no contraindications to synchronized cardioversion (see, for example, Goyal reference cited above), with sufficient sensitivity in the patient monitoring sensors of the AED, along with sufficiently short latency between the patient monitoring sensors, signal processing electronics, shock delivery circuitry, along with sophisticated ECG signal analysis software, it is possible to synchronize the shock delivery with an observed or predicted peak in the heart rhythm, even with VF.

Therefore, in a further embodiment, a sync-enabled AED analyzes the heart rhythm in ventricular fibrillation (VF) and delivers the electrical shock in synchronization with an anticipated or observed peak in the heart rhythm. The analysis may include, for example, monitoring of the patient heart rhythm for determination of shockable rhythm, analysis of general trends and/or average rhythm and rate of the patient heartbeat even during VF. In certain cases, the determination of the presence of a shockable rhythm may be performed separately from the VF analysis, once VF has been observed in the patient. Similar to the VT sync analysis discussed above, the VF analysis may be an integrated part of the heart rhythm analysis performed by the AED in its normal course of electrical shock delivery and may include various types of data analysis integrated into the processing functions of the AED.

It is noted that, when VF is detected, the aim of the synchronized shock delivery may not necessarily be the return of spontaneous circulation (ROSC). Instead, the aim of the shock delivery in a VF patient may be simply a change in the heart rhythm. Ideally, the delivery of an electrical shock via the AED to the VF patient results in a normal sinus rhythm (NSR). If the shock results in VT, SVT, or pulseless VT, the AED may be configured to provide one or more additional electrical shocks to the patient. If the shock delivery results in pulseless electrical activity (PEA), then the patient may continue to be treated, for example, by medication, such as an injection of a vasopressor such as epinephrine, and cardiopulmonary resuscitation (CPR).

As described above with respect to the VT sync analysis, VF sync analysis may be performed alternatively or in addition to the VT sync analysis by monitoring and analysis circuitry already a part of the electrical shock delivery functionality. In this way, no separate input from a user is needed to take advantage of VT and/or VF synchronization in the AED operation. The patient heart rhythm monitoring and analysis is initiated upon application of the AED cardiac pads on the patient, as a normal course of AED operations such that the VT/VF sync function is integrated into the shock delivery operations of the AED.

In certain embodiments, similarly to the AED with integrated VT sync functions, the AED with VF sync includes low latency, high sensitivity monitoring of patient heart rhythms. In certain cases, the same circuitry and analysis processes used in the determination of a shockable rhythm may be used for the VF sync operation. In some cases, the heart rhythm analysis occurs at rates fast enough to monitor heart rhythms to take multiple measurements over typical ECG monitoring timeframes. For instance, the AED with integrated VT and/or VF function may be capable of taking measurements of the heart rhythm at 30 millisecond intervals or less, with enough sensitivity to track trends in the QRS complex to anticipate the peak of the next R wave and/or track trends in the VF measurements.

For example, the VF analysis may include recording the ECG over a predetermined period of time (e.g., 200 milliseconds). Then, the average period between peaks over a given threshold value (e.g., a threshold voltage set by the user or the AED manufacturer at a certain measured voltage on the ECG). The process may then calculate the estimated time of the next anticipated peak, then automatically synchronize the shock delivery with the next anticipated peak.

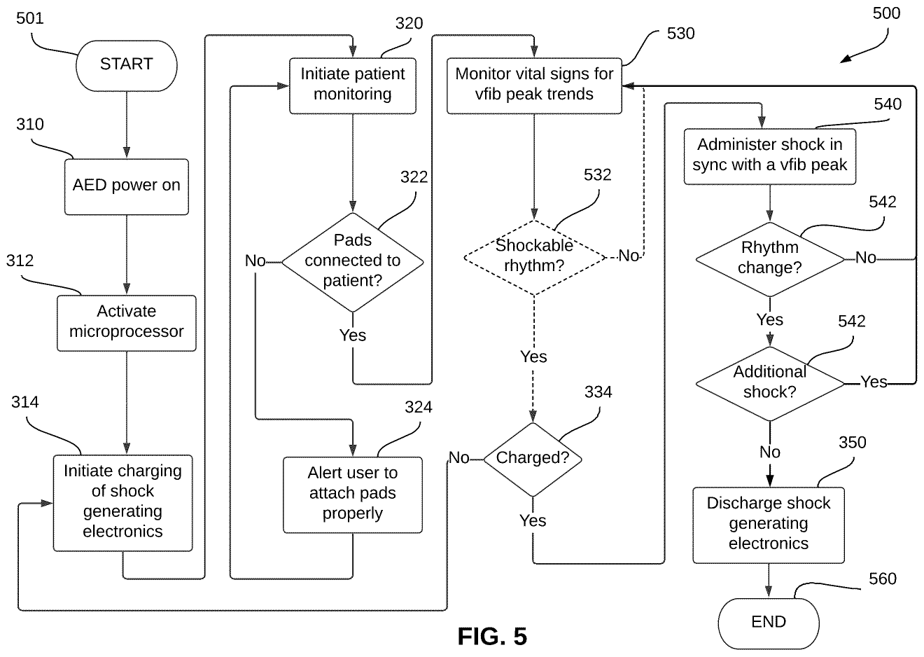

An exemplary process flow for the operation of an AED with integrated VF synchronization is shown in FIG. 5. A process 500 includes several of the same steps as process 300 for AED operations with AED synchronization. In the case of VF synchronization, the determination of shockable rhythm may be an optional decision as VF patients generally exhibit erratic heart rhythm behavior such that determination of the presence of a shockable rhythm may not be possible.

Instead, process 500 begins with a start step 501, then proceeds through the same preparation steps 310, 312, 314, 320, 322, and 324 as with the VT synchronization operation. Process 500 then proceeds to a step 530 to monitor the vital signs of the patient (including heart rhythm) to provide data to the ECG monitoring circuitry, controller, and/or analysis module 245 to analyze trends in the VF peaks. Optionally, a decision 532 is made to determine whether shockable rhythm has been detected. Alternatively, step 530 proceeds directly to decision 334 to determine whether the shock generating electronics are ready for shock delivery.

Once the shock generating electronics have been sufficiently charged, then a shock is delivered in a step 540 in synchrony with the next anticipated VF peak. A decision 542 is then made to determine whether a change in the heart rhythm (e.g., return of spontaneous circulation, VF with different characteristics from prior to the shock delivery, of loss of heart function) has resulted after the shock delivery. If decision 542 determines no change in the heart rhythm has been detected, then process 500 returns to step 530 to continue monitoring the patient vital signs.

If a rhythm change has been detected, then process 500 proceeds to a decision 542 to determine whether additional shock delivery may be required. For example, if the patient heart rhythm now indicates shockable rhythm or a complete loss of pulse, then additional shock delivery may be required. In this case, process 500 again returns to a step 530. If a determination is made that no additional shock delivery would be required, then process 500 proceeds to step 350 to discharge the shock generating electronics, and the process terminates in an end step 560.

In embodiments, an AED with VT sync functions may exhibit low latency from QRS detection to the communication of the detection to the shock delivery mechanism. As an example, a maximum delay from detection of a true R wave peak and the communication of the true R wave peak detection to the shock delivery mechanism is less than 35 milliseconds (e.g., less than a standard ECG readout time interval, or less than half of an average QRS cycle). In an example, the latency may be less than 20 milliseconds. In certain implementations, the median latency may be about 16 milliseconds.

Further, in embodiments, the sensors, firmware, and/or software provide sufficient sensitivity to track trends in the QRS complex and anticipate the peak of the R wave to sync with the shock delivery. For instance, the AED with sync functions includes sensors with sufficient sensitivity to detect electrical signals associated with heart rhythms and provide data of sufficient precision to enable accurate detection of the peak of the R wave, communication of the R wave detection to the shock delivery mechanism, and synchronization of the shock delivery with the immediately following R wave. In an alternative embodiment, the sensing and processing capabilities of the AED with sync functions are able to identify the T wave in the QRS complex and enable delivery of the electrical shock to avoid the T wave and/or cardiac repolarization. In certain embodiments, no user intervention, such as the pressing of a sync button, is required to perform the synchronized cardioversion with the sync-enabled AED.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding”—whether explicitly discussed or not—and, conversely, were there only disclosure of the act of “protruding”, such a disclosure should be understood to encompass disclosure of a “protrusion”. Such changes and alternative terms are to be understood to be explicitly included in the description.

1. An automated external defibrillator system comprising:
shock generating electronics;
an electrocardiogram (ECG) monitoring circuitry; and
a controller for controlling operations of the shock generating electronics and the ECG monitoring circuitry, the controller including non-transitory memory for storing software instructions and a processor for executing software instructions stored on the non-transitory memory,
wherein the controller is configured for receiving and analyzing ECG signals from the ECG monitoring circuitry,
predicting timing of QRS complexes associated with the ECG signals, and
directing the shock generating electronics to deliver an electrical shock in synchrony with the timing of the QRS complexes so predicted.

2. The automated external defibrillator system of claim 1,
wherein the controller is configured for directing the shock generating electronics to deliver the electrical shock in synchrony with an R wave of the QRS complexes so predicted.

3. The automated external defibrillator system of claim 1,
wherein the controller is further configured for
analyzing for ventricular fibrillation (VF) associated with the ECG signals,
predicting a trend in VF peaks associated with the ECG signals, and
directing the shock generating electronics to deliver the electrical shock in synchrony with a VF peak according to the trend in VF peaks so predicted.

4. A method for operating an automated external defibrillator system, the method comprising:
receiving and analyzing electrocardiogram signals from a patient through an electrocardiogram monitoring function in the automated external defibrillator system;
predicting timing of QRS complexes associated with the ECG signals; and
delivering an electrical shock to the patient in synchrony with the timing of the QRS complexes so predicted.

5. A method for operating an automated external defibrillator system, the method comprising:
receiving electrocardiogram (ECG)signals from a patient through an ECG monitoring function in the automated external defibrillator system;
analyzing for ventricular fibrillation (VF) associated with the ECG signals;
predicting a trend in VF peaks associated with the ECG signals; and
delivering the electrical shock to the patient in synchrony with a VF peak according to the trend in VF peaks so predicted.
